(19) United States
(12) Patent Application Publication
(10) Pub. No.: US 2015/0202212 A1
(43) Pub. Date: Jul. 23, 2015

(54) METHODS FOR IDENTIFYING AGENTS FOR TREATING SMOOTH MUSCLE DISORDERS AND COMPOSITIONS THEREOF

(71) Applicant: University of Massachusetts Medical School, Boston, MA (US)

(72) Inventor: Ronghua ZhuGe, Rutland, MA (US)

(73) Assignee: University of Massachusetts Medical School, Boston, MA (US)

(21) Appl. No.: 14/416,273

(22) PCT Filed: Jul. 22, 2013
(86) PCT No.: PCT/US13/51543

§ 371 Date: Jan. 21, 2015

Related U.S. Application Data
(60) Provisional application No. 61/677,674, filed on Jul. 31, 2012.

(51) Int. Cl. A61K 31/575; G01N 33/50

(52) U.S. Cl. CPC A61K 31/575; G01N 33/5061; G01N 33/5038

(57) ABSTRACT

The invention generally relates to methods and compositions for identifying novel therapeutic agents, and uses thereof. More particularly, the invention relates to novel methods for identifying agents useful in treating smooth muscle disorders.

### PRIORITY CLAIMS AND RELATED PATENT APPLICATIONS

This application claims the benefit of priority from U.S. Provisional Application Ser. No. 61/677,674, filed on Jul. 31, 2012, the entire content of which is incorporated herein by reference in its entirety.

### TECHNICAL FIELD OF THE INVENTION

The invention generally relates to methods and compositions for identifying novel therapeutic agents, and related methods of use thereof. More particularly, the invention relates to novel methods for identifying agents, e.g., bitter tastants, which are useful in treating smooth muscle disorders, pharmaceutical compositions comprising such agents and related methods of use.

### BACKGROUND OF THE INVENTION

Airway obstructive diseases, such as asthma and chronic obstructive pulmonary disease (COPD), have become increasingly prevalent, currently affecting more than 300 million people worldwide. Asthma is an obstructive lung disease where the bronchial tubes (airways) are extra sensitive and, when inflamed, can cause muscles around the airways to tighten making the airways narrower. Asthma is usually triggered by dust, pollen and other allergens, upper respiratory tract infections, etc. COPD, also known as chronic obstructive lung disease, is the occurrence of chronic bronchitis or emphysema, a pair of commonly co-existing diseases of the lungs in which the airways become narrowed, which limits air flow to and from the lungs and causes shortness of breath.

Dysfunction of airway smooth muscle (ASM) cells in the respiratory tree plays a pivotal role in promoting progression of airway obstructive diseases and in contributing to their symptoms. (Grainge, et al. 2011 New Engl J Med 364:2006-2015; Hershenson, et al. 2008 Annual Rev Pathol: Mechanisms of Disease 3:523-555; Tliba, et al. 2009 Annual Rev Physiol 71:509-535.) With their ability to contract and relax, smooth muscle cells regulate the diameter and length of conducting airways, controlling dead space and resistance to airflow. Excessive contraction of smooth muscle cells can be life-threatening as they can cause the airway to fully close.

There is an unmet clinical need for new and effective treatments for smooth muscle disorders: (1) their prevalence has almost doubled worldwide in the last few decades, (2) many asthmatics and COPD patients do not respond well to current bronchodilators, and (3) no major breakthrough in bronchodilator development has been achieved since the discovery of specific 132 adrenergic receptor agonists almost fifty years ago.

Bronchodilators have been used to treat asthmatic attacks and to manage COPD. (Fanta 2009 New Engl J Med 360:1002-1014; Han, et al. 2011 Proc of Am Thoracic Soc 8:356-362.) Existing bronchodilators, however, have undesirable side effects and are not sufficiently effective for severe asthmatics and many COPD patients. Understanding the mechanisms regulating ASM holds the promise of developing more effective and safe bronchodilators.

Bitter tastants represent a new class of compounds with potential as potent bronchodilators. Bitter taste, the most sensitive of the five basic tastes, is key to animal and human survival since it help them avoid harmful toxins and noxious substances. Deshpande et al. recently found that cultured ASM cells express G-protein coupled bitter taste receptors (TAS2Rs), a class of proteins long thought to be expressed only in the specialized epithelial cells in the taste buds of the tongue. (Deshpande, et al. 2010 Nat Med 16:1299-1304; Chandrashekar, et al. 2000 Cell 100:703-711; Ruiz-Avila, et al. 1995 Nature 376:80-85; Wong, et al. 1996 Nature 381:796-800; Zhang, et al. 2003 Cell 112:293-301.) Bitter tastants with diverse chemical structures have been shown to cause greater ASM relaxation in vitro than β2 adrenergic agonists, the most commonly used bronchodilators to treat asthma and COPD. (Deshpande, et al. 2010 Nat Med 16:1299-1304; Zhang, et al. 2012 Nat Med 18:648-650.) Moreover, these compounds can effectively relieve in vivo asthmatic airway obstruction than 132 adrenergic agonists in a mouse model of asthma, making them highly attractive bronchondilators for asthma and COPD.

Bitter tastant-induced bronchodilation was unexpected, because these agents appeared to increase intracellular Ca²⁺ concentration ([Ca²⁺]ᵢ) to a level comparable to that produced by potent bronchoconstrictors, which should have led to smooth muscle contraction. (Deshpande, et al. 2010 Nat Med 16:1299-1304; Somlyo, et al. 1994 Nature 372:231-236.) To reconcile this apparent paradox, it was proposed that bitter tastants activate the canonical bitter taste signaling pathway (i.e., TAS2R-gustducin-phospholipase Cβ(PLCβ)-inositol 1,4,5-triphosphate receptor (IP3R)) to increase focal Ca²⁺ release from endoplasmic reticulum, which then activate large-conductance Ca²⁺-activated K⁺ (BK) channels thereby hyperpolarizing the membrane. (Deshpande, et al. 2010 Nat Med 16:1299-1304.) It was, however, subsequently demonstrated through patch-clamp recordings that bitter tastants do not activate BK channels but rather inhibit them. (Zhang, et al. 2012 Nat Med 18:648-650.) Moreover, three different BK channel blockers did not affect the bronchodilation induced by bitter tastants.

The apparent conundrum of putative [Ca²⁺]_{I} elevation leading to relaxation may be attributed to the fact that Ca²⁺ responses to bitter tastants were assessed in cultured human ASM cells, while the contractile responses to them were investigated in freshly dissected ASM tissues. (Deshpande, et al. 2010 Nat Med 16:1299-1304.) It is well known that cultured smooth muscle cell lines alter their phenotype, i.e., losing their ability to contract and relax. (Chamley-Campbell, et al. 1979 Physiol Rev 59:1-61; Hall, et al. 1995 Am J Physiol 268:L1-11.) It is likely their Ca²⁺ response is also modified. To understand bitter tastant-induced bronchodilation, it is necessary to study the contraction and the underlying signaling in freshly isolated ASM tissues and cells.

Thus, in addition to an ongoing need for agents, such as bitter tastants, that are therapeutically effective in treating ASM-related diseases, an urgent need remains for novel methodologies for screening and testing compounds, such as bitter tastants.

### SUMMARY OF THE INVENTION

The invention provides a novel methodology for identifying agents that are useful as therapeutic agents for smooth muscle disorders. The invention also provides pharmaceutical compositions, and methods thereof, useful in preventing, treating or managing smooth muscle disorders.

In one aspect, the invention generally relates to a method for identifying a candidate compound for treating or preventing a smooth muscle disorder. The method includes: (1) contacting a test compound with a cell of a smooth muscle tissue or organ; and (2) measuring the intracellular Ca²⁺ concentration before and after contacting the test compound, whereby a decrease of 30% or greater after contacting the test compound is indicative of the activity of the test compound. In certain preferred embodiments, the method further includes, after contacting a test compound: measuring the cell length before and after contacting with the test compound, wherein an increase of 20% or greater is indicative of the activity of the test compound. In certain preferred embodiments, the test compounds are bitter tastants.

In another aspect, the invention generally relates to a method for treating or preventing a smooth muscle disorder in a mammal, including human. The method includes administering to a subject in need thereof a therapeutically effective amount of a pharmaceutical composition comprising a compound having the structural formula of (I):

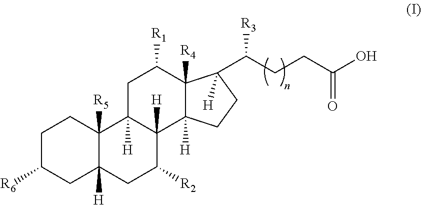

or a pharmaceutically acceptable salt, ester or prodrug thereof, wherein each of R₁, R₂, R₃, R₄, R₅ and R₆ is independently selected from hydrogen, OH, alkyl, alkoxy, and halogen; n is an integer from 0 to about 4.

In yet another aspect, the invention generally relates to a pharmaceutical composition for treating or preventing a smooth muscle disorder in a mammal, including human, comprising a therapeutically effective amount of a pharmaceutical composition comprising a compound having the structural formula of (I):

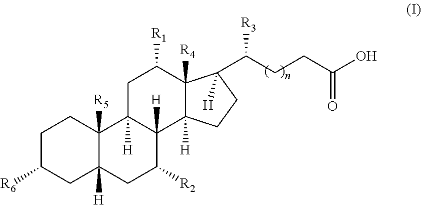

or a pharmaceutically acceptable salt, ester or prodrug thereof, wherein each of R₁, R₂, R₃, R₄, R₅ and R₆ is independently selected from hydrogen, OH, alkyl, alkoxy, and halogen; n is an integer from 0 to about 4.

### BRIEF DESCRIPTION OF THE DRAWINGS

FIG. 1 shows exemplary data demonstrating that bitter tastants induce bronchodilation in a concentration-dependent manner. (i) An original recording of chloroquine (chloro)-induced relaxation of bronchi pre-contracted with 100 μM methacholine (Mch). (ii) Concentration-responses of relaxation caused by denatonium (Denat), quinine, chloroquine, and five bile acids (chenodeoxycholic acid, CDCA; ursodeoxycholic acid, UDCA; deoxycholic acid, DCA; cholic acid, CA; and Lithocholic acid, LCA). As a comparison, the concentration-response to a 13 agonist, isoproterenol (ISO), is also included. To construct these curves, cumulative concentrations of bitter tastents and isoproterenol were administrated to the bronchi pre-contracted with 100 μM Mch.

FIG. 2 shows exemplary data demonstrating that bitter tastants inhibit or potentiate spontaneous tone in mouse internal anal sphincter. (i) An original recording showing bitter tastant (CDCA) reversed totally the spontaneous tone of mouse internal anal sphincter. (ii) A representative recording shows denatonium enhanced the spontaneous tone of mouse internal anal sphincter. The average results on CDCA, UDCA, DCA and chloroquine are shown in (iii). The average results on denatonium are shown in (iv). The spontaneous tone contributes about 70% of the basal tonus that maintains fecal continence in human and animals.

FIG. 3 shows exemplary data demonstrating that bitter tastants relax human bronchi and pulmonary arteries. (i) Bile acid DCA fully relaxed airways pre-contracted by KCl (which makes potential inside membrane more positive, leading to the opening of voltage-dependent Ca²⁺ channels and Ca²⁺ influx). (ii) Chloroquine caused a full relaxation while denatonium failed to exert any effect in human pulmonary arteries pre-contracted by KCl. This also indicates that bitter tastants differentially act on different types of smooth muscle, i.e., their action is specific). (iii) Bile acid DCA markedly relaxed human pulmonary artery pre-contracted by KCl. Human specimen were provided by Univ. Mass. Memorial Medical Center.

FIG. 4 shows exemplary data demonstrating that bitter tastants inhibit mouse urethral contraction induced by contractile agonists. Bitter tastants were pretreated with bitter tastants, and then stimulated with contractile agonists. Contractile agonists alone were used as the control. (A) Chloro (1 mM) blocked 60 mM KCl-induced contraction. (B) CDCA (100 μM) blocked 60 mM KCl-induced contraction. (C) Chloro (1 mM) inhibited alpha1-agonist phenylephrine-induced contraction. (D) 100 μM CDCA inhibited phenylephrine-induced contraction. (E) 100 μM CDCA inhibited 10 μM methacholine-induced contraction.

FIG. 5 shows exemplary data demonstrating that bitter tastants modestly increase intracellular Ca²⁺ concentration ([Ca²⁺]ᵢ) by activating a canonical TAS2R signaling cascade in mouse ASM. (A) Chloroquine (Chloro) raised [Ca²]ᵢ to a level much less than methacholine (Mch). [Ca²⁺]ᵢ was measured with fluo-3 in the form of acetoxymethyl ester, loaded into isolated mouse airway smooth muscle cells, and expressed as ΔF/F₀ (%). (B) 1 mM chloro did not contract airways (using tension as its proxy) while 100 μM Mch caused a robust contraction. Data are mean±s.e.m (n=6 for chloro, and n=5 for Mch). (C) Pertussis toxin (PTX), gallein, Anti-βγ (MPS-phosducin-like protein C terminus, a Gβγ blocking peptide), U73122 and 2-APB inhibited chloro-induced increase in [Ca²⁺]ᵢ (n=19-24 cells). Isolated mouse airway smooth muscle cells were either pretreated with 1 μg/ml PTX for 6-8 hrs or with 1 μM Anti-βγ for 1-2 hrs or with each of the other compounds listed for 5-10 min. The effects of PTX and Anti-βγ were calculated by normalizing the response of chloro to that from the time matched cells without the pretreatments, and the effects of other three compounds were analyzed by normalizing the response of chloro to its own control without the compound. (D) RT-PCR transcripts after amplification with primers to TAS2R107, TAS2R108, α-gustducin, Gβ3, Gγ13, PLCβ2 and β-actin. RNAs were isolated from mouse tracheas and mainstem bronchi, and reactions without complementary DNA were used as a negative control. (E) Cellular distribution of TAS2R107 in three focus planes (bottom, middle, and top) of an isolated mouse airway smooth muscle cell. The TAS2R107 immunostaining intensity after 3D deconvolution (see Methods) was pseudocolored with the color map on the right. This makes positive (but dim) pixels more easily distinguished from background. Eight cells showed a similar subcellular distribution pattern.

FIG. 6 shows exemplary data demonstrating that bitter tastants reverse Mch-induced increase in [Ca²]ᵢ and cell shortening in mouse ASM. (A) Time course of the effect of chloro (1 mM) on a 100 μM Mch-induced increase in [Ca²⁺]ᵢ (represented as ΔF/F₀ integrated over the entire cell) and cell shortening. Images show the changes in [Ca²⁺]ᵢ displayed as fluorescence intensity (rather than ΔF/F₀ to aid visualization). Cell lengths are indicated by red lines. Images were taken at the time indicated on the time course of [Ca²⁺]ᵢ (upper). (B) Relationships between [Ca²⁺]ᵢ (left axis, blue bars) and cell length (right axis, red bars) in response to Mch and Chloro. The letters correspond to the time shown in the upper panel in A (n=23 cells, means±s.e.m; *P <0.05, **P<0.01 using two-tailed Student's t-test). ΔF/F₀ is zero by definition at a, so no blue bar is present at a.

FIG. 7 shows exemplary data demonstrating that suppression of [Ca²⁺]ᵢ by inhibiting L-type VDCCs is necessary for bitter tastant-induced bronchodilation of Mch precontracted mouse airways. (A) Clamping [Ca²⁺]; prevented bitter tastants from causing bronchodilation. Mouse airway strips were permeabilized with α-toxin as described previously (Kitazawa, et al. 1989 J Biol Chem 264:5339-5342), and extracellular [Ca²⁺]ᵢ was set at 1 nM and then switched to 3 μM as indicated in the trace. Seven individual experiments (2 for denatonium, 2 quinine, and 3 chloro all at 1 mM) show responses similar to that shown on the left, so the results were pooled and displayed on the right. Each strip's normalized tension is its tension at the experiment's end divided by its tension just prior to application of bitter tastant, times 100. (B) Left panel: L-type VDCC blocker diltiazem dose-dependently reversed Mch-induced contraction (using tension as a proxy measure) (n=6). Right panel: results for n=6 strips. % relaxation=tension decrease due to diltiazem divided by tension increase due to Mch, times 100. The tension decrease at each concentration of diltiazem is measured once the tension stabilizes. The tension decrease at each increased concentration is always measured relative to the peak tension (i.e., it is total decrease, not the incremental decrease due to the additional diltiazem which was added). (C) FPL 64176 (FPL), a L-type VDCC agonist, prevented chloro from reversing the [Ca²⁺]ᵢ rise induced by Mch. Left panel: a typical time course; ΔF/F₀ for each curve is scaled to have a value of 100 at the peak before chloro is added. Right panel: average results of 16 cells. The values are represented as (ΔF/F₀ at the peak after Mch−ΔF/F₀ at 30 sec after chloro)/(ΔF/F₀ at the peak after Mch−ΔF/F₀ at basal)×100 (i.e., the decrease due to chloro divided by the increase due to Mch). **P<0.01, control vs+FPL. (D) FPL dose-dependently reversed chloro-induced bronchodilation (using tension as a proxy measure) in Mch precontracted airways (n=5-7 independent experiments). Data on the right panels are means±s.e.m. % relaxation definition and analysis are the same as in panel B.

FIG. 8 shows exemplary data demonstrating that KCl only activates L-type VDCCs to increase [Ca²⁺]ᵢ and cause contraction in mouse ASM. (A) KCl failed to generate any global [Ca²⁺]ᵢ increase in the absence of extracellular Ca²⁺. (i) A representative [Ca²⁺]ᵢ response to 60 mM KCl in the presence of extracellular Ca²⁺. (ii, iii, iv), three examples showing that the same concentration of KCl did not increase Ca²⁺ in the zero Ca²⁺ medium. This failure was not due to the depletion of intracellular Ca²⁺ stores because 10 μM Mch still induced Ca²⁺ release either as a single peak or as an oscillation. 8 cells gave rise to similar responses. ΔF/F₀ is the average over the entire cell. (B) KCl (60 mM) caused virtually no increase in tension in the absence of extracellular Ca²⁺. The airways were placed in the Ca²⁺ free solution for 15 min before the measurement commenced. Left panel shows a pair of representative recordings and right panel the average results. **, P<0.01, Student's paired t-test, n=6 independent experiments. (C) KCl (60 mM)-induced increase in [Ca²⁺]ᵢ was blocked by prior application of L-type VDCC blocker diltiazem (100 μM). **, P<0.01, Student's paired t-test, n=9 for each conditions. (D) Diltiazem relaxed KCl-induced contraction of mouse airways. Data are means±s.e.m. (n=6 independent experiments), and % relaxation definition and analysis are the same as in FIG. 7B.

FIG. 9 shows exemplary data demonstrating that bitter tastants block L-type VDCCs. (A) Chloro, denatonium and diltiazem relaxed KCl-induced contraction of mouse airways. Left panel shows representative force recordings in response to KCl followed by chloro and denatonium, and the right the mean values of the relaxation of KCl-induced contraction by chloro, denatonium and diltialzem (n=6-9 independent experiments). (B) Relationship between [Ca²⁺]ᵢ and cell length in response to KCl and chloro. Left panel shows the time course of concomitant changes in [Ca²⁺]ᵢ and cell length and the right the means±s.e.m (n=15 cells) at four time points marked on the left. (C) FPL dose-dependently inhibited chloro-induced bronchodilation of KCl precontracted airways. Left panel shows two representative recordings, and the right panel the means±s.e.m (n=5-7 independent experiments). Given the non-monotonic nature of the relaxation (left), both the greatest reduction in force after chloro (i.e., Maximum) and the force reduction 5 min after chloro were measured and divided by the peak force-resting force before application of chloro. (D) FPL 64176 (FPL) inhibited chloro-induced suppression of the rise in [Ca²⁺]ᵢ produced by KCl. Left panel shows original recordings of Ca²⁺ responses and the right panel the means±s.e.m (n=28 without FPL, n=16 with FPL). The values represent as (ΔF/F₀ at the peak after KCl−ΔF/F₀ at 30 sec after chloro)/(ΔF/F₀ at the peak after KCl−ΔF/F₀ at basal)×100. (E) Chloro blocked L-type VDCC currents. Left panel displays patch clamp recordings of L-type Ca²⁺ currents in response to a voltage pulse from −70 mV to 0 mV in the control and in the presence of 1 mM Chloro, and the right panel the effect of chloro on the current-voltage (I-V) relationship of the Ca²⁺ current (n=5). Ba²⁺ was used as a charge carrier, and the peak current was used to construct the I-V relationship. The high voltage threshold for activation seen in the I-V relationship, and its sensitivity to FPL and nifedipine indicate these Ca²⁺ currents resulted from the opening of L-type VDCCs. *P<0.05; **P<0.01. (Zhuge, et al. 2010 J Biol Chem 285:2203-2210.)

FIG. 10 shows exemplary data demonstrating that bitter tastants inhibit L-type VDCCs via a Gβγ dependent process. (A) Representative recordings of changes in [Ca²⁺] in response to KCl followed by chloro (I mM) with and without pretreatment with PTX (1 μg/ml), gallein (1 μM), anti-βγ blocking peptide, U73122 (3 μM) and 2-APB (50 μM). The application protocols for these compounds were the same as in the experiments in FIG. 5C. All data were scaled to have a maximum of 100 and aligned at the time point when KCl was administrated. (B) Effects of compounds listed in A on chloro-induced suppression of KCl-induced increase in [Ca²⁺]ᵢ. The values were calculated the same as in FIG. 8D. Compared to the control (i.e., chloro alone after KCl, FIG. 8D), P<0.0001 for PTX, gallein, and anti-βγ; and P>0.05 for U73122 and 2-ABP. Data are shown as means±s.e.m (n=12-38 cells). (C) A model for TAS2R signaling and bitter tastant-induced bronchodilation.

FIG. 11 shows exemplary data demonstrating that bitter tastant chloroquine dose-dependently increased [Ca²⁺]ᵢ in resting single cells (A) without a significant effect on the contractility (B) of relaxed mouse airways. Results are mean±s.e.m, (n=5-30 cells in A and 7 airways in B). Dose response in A was generated based on the responses to single dose administration, while that in B was based on accumulative administration. Mch produced a much larger response.

FIG. 12 shows exemplary characteristics of [Ca²⁺]ᵢ and contractile responses to bitter tastants and diltiazem in human airway smooth muscle. (A) Bitter tastants reversed the [Ca²⁺]ᵢ rise and cell shortening induced by Mch. Measurements were taken at the steady state levels in response to Mch and chloroquine. The cell length before stimulation was considered as 100%. *, P<0.05 paired student's t-test; ***. P<0.001; n=6-12. (B) L-type VDCC blocker diltiazem dose-dependently reversed 10 μM Mch-induced contraction (n=5 independent experiments). % relaxation=tension decrease due to chloroquine or diltiazem divided by tension increase due to KCl or Mch, times 100. The tension decrease at each concentration of diltiazem is measured once the tension stabilizes. The tension decrease at each increased concentration is always measured relative to the peak tension (i.e., it is total decrease, not the incremental decrease due to the additional diltiazem which was added). (C) Chloroquine and diltiazem relaxed human intrapulmonary bronchi precontracted by 60 mM KCl (n=3-5 independent experiments). Bar charts are means±s.e.m.

FIG. 13 shows exemplary data demonstrating that Ca²⁺ influx plays a major role in producing and maintaining a Mch-induced increase in [Ca²⁺]ᵢ and contraction in mouse ASM. (A) In Ca²⁺ free medium (n=10 airways), the tension generated by Mch was less than 20% of that in the presence of extracellular Ca²⁺. ***, P<0.001, Student's paired t-test, n=9 airways. (B) Mch increased [Ca²⁺]ᵢ less in Ca²⁺ free medium (n=12 cells) than in the presence of extracellular Ca²⁺ (n=9 cells). In the absence of extracellular Ca²⁺, Mch (10 μM) produced different patterns of changes in [Ca²⁺]ᵢ, so the area under each curve was calculated for one minute of Mch stimulation and compared between the two conditions (right panel). ***, P<0.001 with Ca²⁺ vs without Ca²⁺, Student's unpaired t-test, n=11. (C) Ca²⁺ stores remained functional in the absence of extracellular Ca²⁺. The cells were placed in the absence of extracellular Ca²⁺ for 15 min, and then stimulated with two Mch pulses 15 min apart. The chart on the right indicates that two Mch administrations produced comparable Ca²⁺ response, i.e., Ca²⁺ stores are intact under experimental conditions in the present study. N.S, P>0.05 for the response in the first pulse of Mch vs that in the second pulse, Student's paired t-test, n=10. ΔF/F₀ for B and C are the average over the entire cell.

FIG. 14 shows exemplary data demonstrating that bitter tastants reverse contractile agonist-induced increase in [Ca²⁺]ᵢ in isolated smooth muscle cells from mouse internal anal sphincter. (A) CDCA (100 μM) reversibly inhibited the increase in [Ca²⁺]ᵢ induced by 60 mM KCl. (B) Chloroquine (1 mM) fully reversed the increase in [Ca²⁺]ᵢ induced by 60 mM KCl. (C) Denatonium (1 mM) did not affect the [Ca²⁺]ᵢ response to 60 mM KCl.

### DETAILED DESCRIPTION OF THE INVENTION

The present invention provides novel methodologies for screening and testing compounds, such as bitter tastants. The unique approach disclosed herein is based on a better understanding of the underlining mechanisms regulating ASM. The invention also provides pharmaceutical compositions and methods of use of certain bitter tastants that are therapeutically effective in treating ASM-related diseases.

Smooth muscles express bitter taste receptors, the activation of which induces profound changes in the contractility of smooth muscle. Bitter taste receptors are believed to be targets for treating diseases or disorders in smooth muscle. Bitter compounds represent a good starting point for developing therapeutics that relax airway smooth muscle more effective than β₂ agonists, the commonly used bronchidilators for airway obstructive diseases. Some bitter compounds may be capable of relaxing bronchoconstrictor pre-contracted airways that are resistant to β₂ agonist treatment. A unique and effective screening methodology has been developed and disclosed herein that is promised to change the paradigm of research and development on smooth muscle disorders.

As disclosed first herein, bitter tastants activate the canonical bitter taste signaling cascade, slightly increasing global the intracellular Ca²⁺ concentration ([Ca²⁺]ᵢ) in resting cells, but not to a level sufficient to cause contraction. However, bitter tastants reverse the increase in [Ca²⁺]ᵢ evoked by bronchoconstrictors, leading to bronchodilation. This reversal is mediated by the suppression of L-type voltage-dependent Ca²⁺ channels (VDCCs) in a gustducin βγ subunit-dependent, yet PLCβ- and IP3R-independent manner. Hence, it is believed that TAS2R activation in ASM stimulates two opposing Ca²⁺ signaling pathways, both mediated by Gβγ subunits, which increases [Ca²⁺]ᵢ at rest but blocks activated L-type VDCCs reversing the contraction they cause. Therefore, bitter tastants can generate different and opposing Ca²⁺ signals depending upon the cellular environment.

The present invention revealed two major differences in Ca²⁺ signaling compared to a prior study by Deshpande et al. (Deshpande, et al. 2010 Nat Med 16:1299-1304.) First, Desphande et al. reported that bitter tastant increased [Ca²⁺]ᵢ to a level comparable to bronchoconstictors. In freshly isolated ASM, we found that bitter tastants only modestly increased [Ca²⁺]ᵢ to a level much lower than that produced by bronchconstrictors. Second, Deshpande et al. reported that bitter tastants generate local Ca²⁺ events. In freshly isolated ASM, in contrast, we found that bitter tastants did not increase local Ca²⁺ releases such as Ca²⁺ puffs and Ca²⁺ sparks. A reason for these discrepancies may be because Despande et al.'s studies were conducted in cultured ASM cell lines, as oppose to freshly isolated ASM, which display a different phenotype by altering the expression of receptors, ion channels and contractile proteins. (Chamley-Campbell, et al. 1979 Physiol Rev 59:1-61; Hall, et al. 1995 Am J Physiol 268:L1-11.) Additionally, we found that bitter tastants do not activate BK channels. Thus, the evidence establish that bitter tastant-induced bronchodilation is highly unlikely to result from the generation of local Ca²⁺ events, which in turn activate BK channel and hyperpolarize the membrane as proposed previously. (Deshpande, et al. 2010 Nat Med 16:1299-1304.)

By simultaneously measuring [Ca²⁺]ᵢ and cell shortening, we found that bitter tastant's ability to reverse the increase in [Ca²⁺]ᵢ caused by bronconstrictors is the underlying signal producing the bronchodilation. The conclusion that [Ca²⁺]ᵢ is the critical signal governing ASM contractility was supported by at least three lines of evidence. First, in the presence of bronchoconstrictors, bitter tastants lowered [Ca²⁺]ᵢ while at the same time relaxing the precontracted cells. This response was found to be reversible. Second, clamping intracellular [Ca²⁺]ᵢ to levels produced by the bronchoconstrictors (low μM) prevented bitter tastants from relaxing airways. Third, enhancing and blocking Ca²⁺ influx via L-type Ca²⁺ channels oppositely regulated the relaxation mediated by bitter tastants.

Gustducin Gβγ inhibits L-type VDCCs to cause bronchodilation, highlighting the importance of these channels in mediating bronchoconstriction and their potential as a target for bronchodilators. Indeed, L-type VDCCs are expressed in ASM cells and their activation causes these cells to fully contract. (Du, et al. 2006 J Biol Chem 281:30143-30151; Kotlikoff 1988 Am J Physiol 254:C793-801; Liu, et al. 2006 Am J Physiol Lung Cell Mol Physiol 291:L281-288; Zhuge, et al. 2010 J Biol Chem. 285:2203-2210.) Activation of these channels is a key mechanism underlying bronchoconstrictor-induced contraction. (Gosens, et al. 2006 Respiratory Res 7:73; Hirota, et al. 2003 British J Anaesthesia 90:671-675; Kajita, et al. 1993 Am J Physiol 264:L496-503; Liu, et al. 2006 Am J Physiol Lung Cell Mol Physiol 291:L281-288.) Moreover, antagonists of L-type VDCCs are effective in relieving airway spasm in animal models of asthma and in at least a subset of asthmatic patients. (Ahmed, et al. 1988 J Allergy & Clinical Immunol 81:133-144; Barnes, et al. 1981 Thorax. 36:726-730; Harman, et al. 1987 Am Rev Respir Dis 136:1179-1182; Patel, et al. 1985 Eur J Respir Dis. 67:269-271.) Although L-type VDCCs in smooth muscle can be modulated by a variety of means including phosphorylation and Ca²⁺, this is the first demonstration that a Gβγ can inhibit L-type VDCCs in smooth muscle. (Gui, et al. 2006 J Biol Chem 281:14015-14025; Le Blanc, et al. 2004 Circulation Res 95:300-307; Liao, et al. 2005 Cardiovascular Res 68:197-203; Thakali, et al. 2010 Circulation Res 106:739-747; Zhong, et al. 2001 J Physiol 531:105-115.) Given that Gβγ can directly inhibit K⁺ channels and N type Ca²⁺ channels in several cell types (Herlitze, et al. 1996 Nature 380:258-262; Ikeda 1996 Nature 380:255-258; Reuveny, et al. 1994 Nature 370:143-146; Wickman, et al. 1994 Nature 368:255-257), it is likely that Gβγ acts on L-type VDCCs in a similar manner.

The opposing Ca²⁺ signals mediated by Gβγ upon activation of G-protein coupled bitter taste receptors (TAS2Rs) revealed in this study are unique. It is expected that gustducin Gβγ activates PLCβ to generate IP3 and release Ca²⁺ from endo/sarcoplasmic reticulum to raise [Ca²⁺]ᵢ in ASM cells. But, unexpectedly, gustducin Gβγ also suppresses Ca²⁺ signaling mediated by Mch, which largely activates M3 muscarinic acetylcholine receptor, a Gq family receptor. In general, Gβγ from the Gᵢ/Gₒ family (to which TAS2Rs belong) tends to potentiate, rather than, inhibit the Ca²⁺ responses caused by the Gq family. (Cheng, et al. 2002 Biochem J 364:33-39; Samways, et al. 2003 Biochem J 375:713-720.) It remains to be determined whether the inhibition of Ca²⁺ signaling by TAS2R activation is Gβγ isoform specific. Since Gβγ also mediates the ASM contractions induced by activation of M2 muscarinic acetylcholine receptors and γ-aminobutyric acid-B receptors, our present findings suggested that Gβγ reversal of the rise in [Ca²⁺]_{I} caused by bronchoconstrictors is isoform specific, and is likely via Gβ3γ13 dimers which are released upon activation of TAS2Rs. (Mizuta, et al. 2011 Am J Respiratory Cell and Mol Biol 45:1232-1238; Nino, et al. 2012 PLoS ONE 7:e32078; Huang, et al. 1999 Nat Neurosci 2:1055-1062.)

Investigation was directed at how bitter tastants affected both [Ca²⁺]ᵢ and ASM contraction in freshly isolated airway cells and tissue from mouse and human. Fluo-3 was used to assess the effect of bitter tastants on [Ca²⁺]ᵢ. Chloroquine and denatonium, two substances commonly used to study bitter taste signaling, were used as bitter tastants.

It is worth mentioning that virtually all of the studies of bitter taste signaling in taste buds and extraoral tissues have focused on the responses mediated by bitter tastants alone. (e.g., Chandrashekar, et al. 2000 Cell 100:703-711; Ruiz-Avila, et al. 1995 Nature 376:80-85; Wong, et al. 1996 Nature 381:796-800; Zhang, et al. 2003 Cell 112:293-301; Janssen, et al. 2011 Proc of Nat Academy of Sci 108:2094-2099; Shah, et al. 2009 Science 325:1131-1134; Tizzano, et al. 2010 Proc of Nat Academy of Sci 107:3210-3215.) The opposing Ca²⁺ signaling mediated by Gβγ as disclosed herein may operate in these systems when they are stimulated by a combination of bitter tastants and other activators.

Bitter tastants induce a stronger bronchodilation in both in vitro and in vivo asthmatic mouse models than do β2 agonists, the most commonly used bronchodilators for treating asthma and COPD. Therefore, these compounds are promising candidates to be developed as a new class of bronchodilators. The findings in the present study provide the cellular and molecular rationale for this line of inquiry. Searching for these bitter tastants is of clinical significance because the current bronchodilators are insufficient for treating severe asthma and many COPD patients.

TAS2Rs had long been thought to function only in specialized epithelial cells in the taste buds of the tongue. However, studies in recent years have demonstrated that activation of TAS2Rs can generate different biological responses in a variety of extraoral tissues. Bitter tastants were found to cause airway smooth muscle relaxation in vitro in normal mice and human lung specimens, and in vivo in asthmatic mice. This relaxation is greater than that produced by β2 adrenergic agonists, the most commonly used bronchodilators for symptomatic relief in asthma and chronic obstructive pulmonary disease.

To uncover the underlining mechanism, freshly isolated airway tissue and airway smooth muscle cells from mice and humans have been studied using a combination of Ca²⁺ imaging, patch-clamp recording, single cell shortening/tissue contraction assay, and pharmacology. The results showed that activation of TAS2Rs in airway smooth muscle releases the G-protein gustducin βγ. Surprisingly, gustducin βγ, on the one hand, mediates an modest elevation in intracellular Ca²⁺ concentration ([Ca²⁺]ᵢ) in resting cells and, on the other hand, reverses the rise in [Ca²⁺]ᵢ seen in cells treated with bronchoconstrictors (e.g. Gq-coupled receptor agonists), meant to simulate asthma, by suppressing L-type voltage-dependent Ca²⁺ channels, thereby producing relaxation.

Disclosed first herein is that Gβγ mediates opposing Ca²⁺ signaling mechanisms, which uncovers a new form of signaling that integrates two major cellular signaling systems (i.e., G-protein coupled receptor and Ca²⁺) since Gβγ from the Gᵢ/Gₒ family usually potentiates, rather than, inhibits the responses by the Gq family. This mechanism likely operates in many types of smooth muscle—a tissue essential for virtually all the internal hollow organs in animals and human, and involved in an array of diseases or disorders such as hypertension and overactive bladder.

The present invention provides (1) a cellular and molecular basis of a new form of bronchodilation, bitter tastant-induced bronchodialtion, and (2) a molecular explanation for a new class of bronchodilators potentially better than β2 adrenergic agonists. More importantly from a drug development perspective, the invention reveals a Ca²⁺ effect that is large enough to be well suited for screening and identifying potent bronchodilators from among the many thousands of available bitter tastants. A critical step in identifying highly potent bitter tastants is developing reliable and highly effective screening methodologies. The better understanding of Ca²⁺ dynamics in response to bitter tastants enables a methodology that is promised to accelerate screening and identification of potent bronchodilators from a new class of compounds. Measurements of [Ca²⁺]ᵢ (and optionally in conjunction with measurement of cell shortening) as disclosed herein provide a robust and quantitative approach that represents a powerful new paradigm for identifying bronchodilators from among the many bitter tastants available. Furthermore, the invention provides promising candidates for treating asthma and COPD.

Thus, in one aspect, the invention generally relates to a method for identifying a candidate compound for treating or preventing a smooth muscle disorder. The method includes: (1) contacting a test compound with a cell of a smooth muscle tissue or organ; and (2) measuring the intracellular Ca²⁺ concentration before and after contacting the test compound, whereby a decrease of 30% or greater after contacting the test compound is indicative of the activity of the test compound.

In certain preferred embodiments, the intracellular Ca²⁺ concentration decreased 40% or greater after contacting the test compound is indicative of the activity of the test compound. In certain preferred embodiments, the intracellular Ca²⁺ concentration decreased 50% or greater after contacting the test compound is indicative of the activity of the test compound. In certain preferred embodiments, the intracellular Ca²⁺ concentration decreased 60% or greater after contacting the test compound is indicative of the activity of the test compound. In certain preferred embodiments, the intracellular Ca²⁺ concentration decreased 70% or greater after contacting the test compound is indicative of the activity of the test compound. In certain preferred embodiments, the intracellular Ca²⁺ concentration decreased 80% or greater after contacting the test compound is indicative of the activity of the test compound. In certain preferred embodiments, the intracellular Ca²⁺ concentration decreased 90% or greater after contacting the test compound is indicative of the activity of the test compound.

In certain preferred embodiments, the method further includes, after contacting a test compound: measuring the cell length before and after contacting with the test compound, wherein an increase of 20% or greater is indicative of the activity of the test compound. In certain preferred embodiments, the method further includes, after contacting a test compound: measuring the cell length before and after contacting with the test compound, wherein an increase of 25% or greater is indicative of the activity of the test compound. In certain preferred embodiments, the method further includes, after contacting a test compound: measuring the cell length before and after contacting with the test compound, wherein an increase of 30% or greater is indicative of the activity of the test compound. In certain preferred embodiments, the method further includes, after contacting a test compound: measuring the cell length before and after contacting with the test compound, wherein an increase of 35% or greater is indicative of the activity of the test compound. In certain preferred embodiments, the method further includes, after contacting a test compound: measuring the cell length before and after contacting with the test compound, wherein an increase of 40% or greater is indicative of the activity of the test compound.

In certain preferred embodiments, the test compounds are selected from bitter tastants.

In certain preferred embodiments, the smooth muscle tissue or organ is part of the respiratory tract. In certain preferred embodiments, the smooth muscle tissue or organ is part of a blood vessel. In certain preferred embodiments, the smooth muscle tissue or organ is part of the gastrointestinal tract. In certain preferred embodiments, the smooth muscle tissue or organ is part of the urinary tract. In certain preferred embodiments, the smooth muscle tissue or organ is part of internal anal sphincter. In certain preferred embodiments, the smooth muscle tissue or organ is part of pulmonary artery.

The invention is also directed at compounds identified, via the disclosed methods, to have activity in treating or preventing a smooth muscle disorder.

In another aspect, the invention generally relates to a method for treating or preventing a smooth muscle disorder in a mammal, including human. The method includes administering to a subject in need thereof a therapeutically effective amount of a pharmaceutical composition comprising a compound having the structural formula of (I):

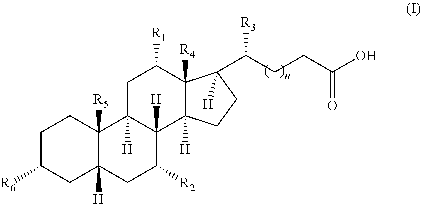

or a pharmaceutically acceptable salt, ester or prodrug thereof, wherein each of R₁, R₂, R₃, R₄, R₅ and R₆ is independently selected from hydrogen, OH, alkyl, alkoxy, and halogen; n is an integer from 0 to about 4 (e.g., 0, 1, 2, 3, 4).

In yet another aspect, the invention generally relates to a pharmaceutical composition for treating or preventing a smooth muscle disorder in a mammal, including human, comprising a therapeutically effective amount of a pharmaceutical composition comprising a compound having the structural formula of (I):

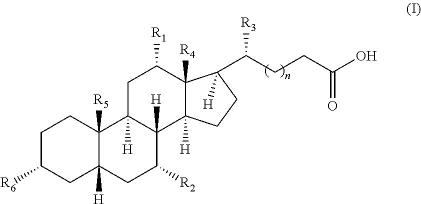

or a pharmaceutically acceptable salt, ester or prodrug thereof, wherein each of R₁, R₂, R₃, R₄, R₅ and R₆ is independently selected from hydrogen, OH, alkyl, alkoxy, and halogen; n is an integer from 0 to about 4 (e.g., 0, 1, 2, 3, 4).

In certain preferred embodiments, the compound has the structural formula:

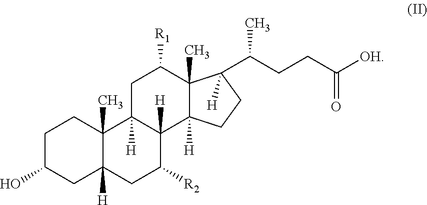

In certain preferred embodiments, the smooth muscle disorder is an airway obstructive disease. In certain preferred embodiments, the airway obstructive disease is asthma. In certain preferred embodiments, the airway obstructive disease is chronic obstructive pulmonary disease (COPD). In certain preferred embodiments, the smooth muscle disorder is anal sphincter disorder. In certain preferred embodiments, the smooth muscle disorder is urethral obstruction. In certain preferred embodiments, the smooth muscle disorder is associated with cystic fibrosis. In certain preferred embodiments, the smooth muscle disorder is associated with fecal incontinence and constipation. In certain preferred embodiments, the smooth muscle disorder is associated with pulmonary hypertension.

In certain preferred embodiments, R₁ is hydrogen. In certain preferred embodiments, R₁ is OH. In certain preferred embodiments, R₂ is hydrogen. In certain preferred embodiments, R₂ is OH.

In certain preferred embodiments, the compound is:

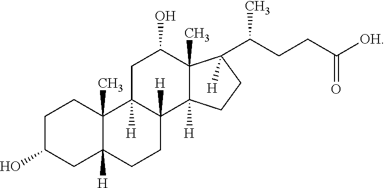

In certain preferred embodiments, the compound is:

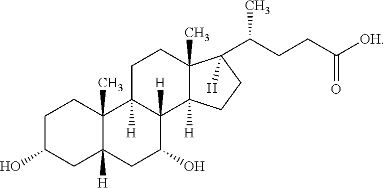

It should be noted that, although the disclosed screening methods are especially suited for bitter compounds, they can be adopted to effectively screen non-bitter compounds. Additionally, bitter compounds may be of a variety of chemical structures and may be naturally occurring or synthetic. Exemplary bitter compounds include those found at: http://en.wikipedia.org/wiki/Category:Bitter_compounds (accessed on Jul. 19, 2012) and derivatives and analogs thereof. For example:

Aloin

Alpha acid

Amarogentin

Andrographolide

Bitrex

Brucine

Caffeine

Denatonium

Eugenin

Hesperidin

Humulone

Isohumulone

Kaempferol 3-O-rutinoside

6-Methoxymellein

Naringin

Papaverine

Phenylthiocarbamide

Propylthiouracil

Exemplary pharmaceutical compositions of the invention include: lung aerosol compositions to prevent, treat or manage asthma, chronic obstructive pulmonary disease, cystic fibrosis; topical preparations to prevent, treat or manage fecal incontinence, haemorrhoids and anal fissure; compositions suitable for oral administration to prevent, treat or manage fecal incontinence and constipation; compositions suitable for oral administration or injection to prevent, treat or manage pulmonary hypertension; compositions suitable for oral administration or injection to prevent, treat or manage urethral obstruction.

### Examples

### Bitter Tastants Modestly Raise Global [Ca²⁺]ᵢ with No Change in Force Generation in Native ASM at Rest

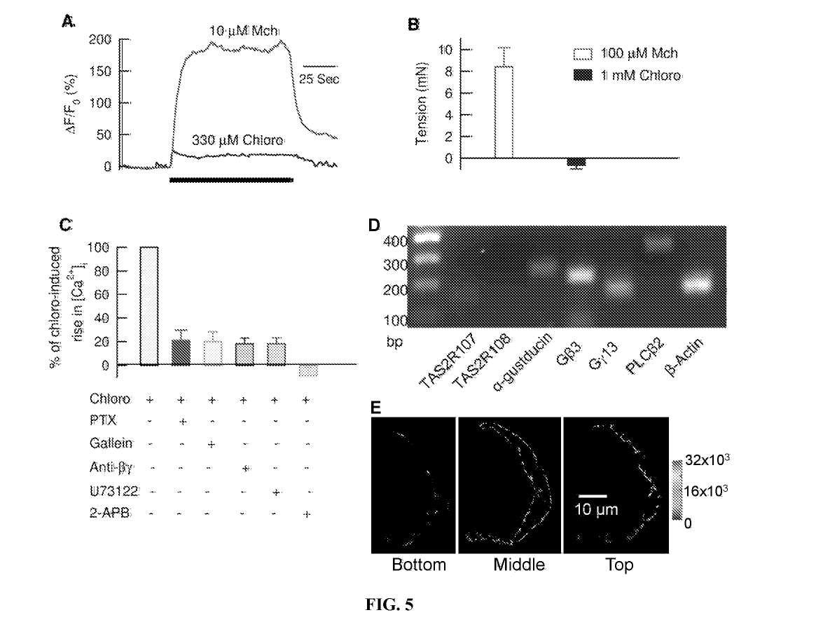

Ca²⁺ response to bitter tastants in resting cells was examined. In contrast to the marked increase in global [Ca²⁺]ᵢ reported in resting cultured human ASM cells (Deshpande, et al. 2010 Nat Med 16:1299-1304), we observed, in resting native ASM from mouse, that chloroquine (0.1 mM) only modestly raised global [Ca²⁺]ᵢ (and to a level much lower than when cells contracted after application of Mch at 0.01 μM-100 μM) (FIG. 5A and FIG. 11A). Chloroquine (330 μM) increased fluo-3 fluorescence (ΔF/F₀) (i.e., [Ca²⁺]ᵢ) both in the presence of extracellular Ca²⁺ (37.7±8%, n=19) and in its absence (29.3±6%, n=15; P>0.05), indicating that the source for this chloroquine response is from internal Ca²⁺ stores.

To examine whether this modest increase in [Ca²⁺]ᵢ is sufficient to trigger contraction, smooth muscle force formation in mouse airways was measured. As shown in FIG. 5B and FIG. 11B, chloroquine (10 μM-1 mM) did not cause contraction of mouse airways, although there was a tendency to decrease the basal tone of airways. As a comparison, Mch at concentrations between 0.1 μM and 10 μM induced contraction markedly and in a dose-dependent manner (FIG. 5B and FIG. 11B).

### Bitter Tastants do not Generate Localized Ca²⁺ Events

Mouse ASM cells exhibit spontaneous Ca²⁺ sparks resulting from the opening of ryanodine receptors in the sarcoplasmic reticulum (Zhuge, et al. 2010 J Biol Chem 285:2203-2210). To test whether bitter tastants generate local Ca²⁺ events as proposed by others (Deshpande, et al. 2010 Nat Med 16:1299-1304), ASM cells were stimulated with chloroquine (10 μM, a concentration around EC50) for 2 mins and measured Ca²⁺ sparks. Off 40 chloroquine-stimulated cells, 27 cells generated a global [Ca²⁺]ᵢ increase that precluded an accurate estimate of Ca²⁺ sparks. In the remaining 13 cells without a detectable global rise in [Ca²]ᵢ, chloroquine inhibited the spark frequency but had no effect on the amplitude (Frequency (Hz): 2.13±0.24 in control and 1.62±0.21 with chloroquine (n=13, P<0.05, paired student's t-test); Amplitude (ΔF/F₀ at the brightest location): 20.6±1.69 in control and 18.1±1.3 with chloroquine (n=13, P>0.05, paired student's t-test)). To test whether spontaneous Ca²⁺ sparks mask the effect of bitter tastants on other forms of local Ca²⁺ releases, such as Ca²⁺ puffs due to the opening of IP3Rs (Smith, et al. 2009 Proceedings the Nat Academy of Sci 106:6404-6409), the Ca²⁺ responses to chloroquine in ASM cells pretreated with 100 μM ryanodine was examined. In these cells, prior to chloroquine application, no spontaneous sparks were observed (n=14). Chloroquine (10 μM) increased global [Ca²⁺] by 12±4% (ΔF/F₀ at its brightest location) in 9 cells, and failed to cause any detectable Ca²⁺ increase in 5 cells. There were no detectable local Ca²⁺ events produced in any of the 14 cells. These results indicate that chloroquine at 10 μM does not increase local Ca²⁺ events (either Ca²⁺ puffs or Ca²⁺ sparks).

### Bitter Tastants Activate the TAS2R Signaling Pathway to Modesty Raise Global [Ca²⁺]ᵢ in Native ASM at Rest

Next examined was the cause of the modest global [Ca²⁺]ᵢ rise by bitter tastants. Since in taste cells, bitter tastants bind to TAS2R to activate the pertussis toxin (PTX) sensitive G-protein gustducin, which in turn induces a PLCβ2 and IP3 signaling cascade (Hoon, et al. 1995 Biochem J 309 (Pt 2):629-636; Spielman, et al. 1996 Am J Physiol 270:C926-931), it was studied whether bitter tastants activate this TAS2R signaling pathway. In native ASM cells, PTX (1 μg/ml, and 6-8 hr pretreatment), reduced the chloroquine-induced increase in global [Ca²⁺]ᵢ to 21.1±8.6% of the control cells (n=20; FIG. 5C). Also both gallein (20 μM and 30 min pretreatment), a blocker of the Gβγ dimer of PTX sensitive G proteins, and MPS-phosducin-like protein C terminus, a Gβγ blocking peptide (Anti-βγ; 1 μM, and 1 hr pretreatment) (Morrey, et al. 2008 J Pharmacol and Exp Therap 326:871-878; On, et al. 2002 J Biol Chem 277:20453-20460) reduced the bitter tastant-mediated increase in [Ca²⁺]ᵢ to 19.9±8.5% (n=19; FIG. 5C) and 18.4±4.8% of the controls, respectively. Finally, U73122 (3 μM), a blocker of PLCβ, and 2-Aminoethoxydiphenyl borate (2-APB) (50 μM), an IP3R antagonist, suppressed the bitter tastant-induced increases in [Ca²⁺]ᵢ to 18.0±5.5% (n=24) and −10.5±7.3% of controls, respectively (FIG. 5C). These results indicate that bitter tastants do activate the BT2R signaling transduction pathway (i.e., TAS2R-PTX-sensitive G protein-PLCβ-IP3R) to release Ca²⁺ from internal stores. This conclusion is further supported by the finding that mouse ASM express transcripts for TAS2R107, α-gustducin, Gβ3, Gγ13, and PLCβ2 (FIG. 5D).

Bitter Tastant-Induced Bronchodilation is Due to Reversal of the Rise in Global [Ca²⁺]ᵢ Caused by Bronchoconstrictors

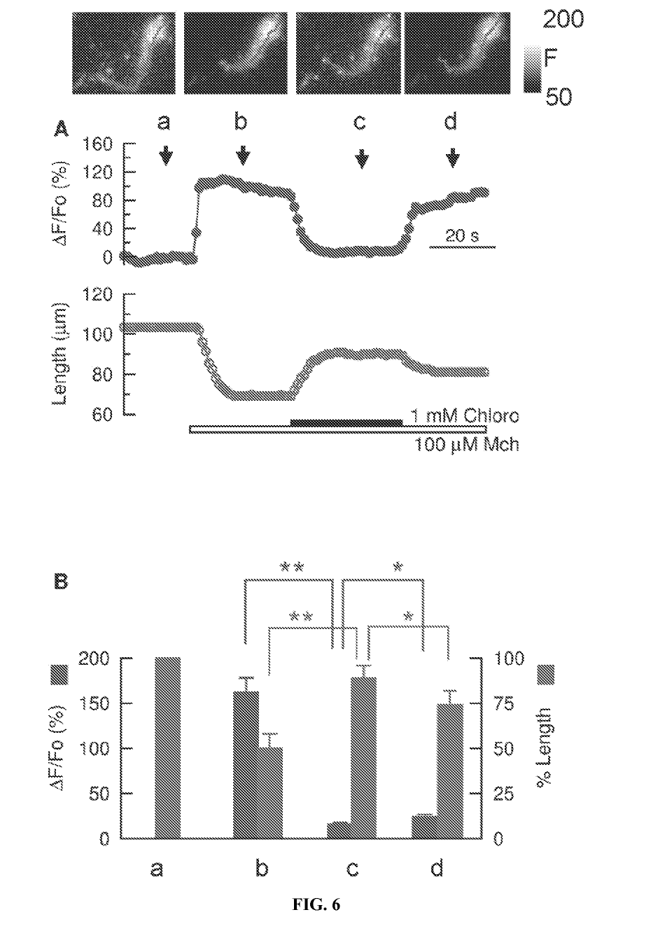

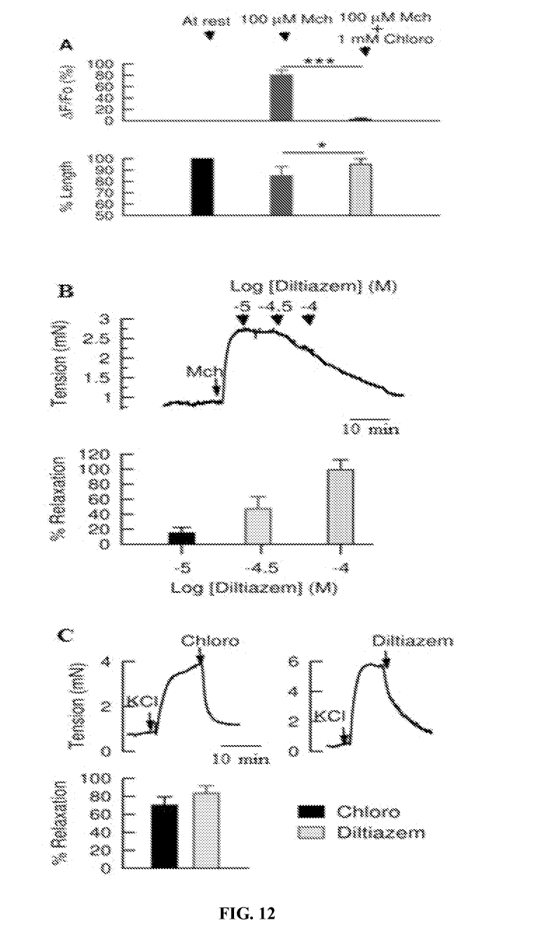

Bitter tastants at μM levels can modestly increase [Ca²⁺]ᵢ in resting cells, but this raises a conundrum as they also can fully relax airways precontracted by bronchoconstrictors. (Deshpande, et al. 2010 Nat Med 16:1299-1304; Zhang, et al. 2012 Nat Med 18:648-650.) In light of the fact that an increase in [Ca²⁺]ᵢ is the primary signal for contraction in all smooth muscle, we explored how bitter tastants affect [Ca²⁺]ᵢ evoked by bronchoconstrictors. To better quantify these effects, we measured ASM Ca²⁺ response and cell shortening at the same time. The cells were stimulated with methacholine (Mch), a stable analogue of acetylcholine that is the major neurotransmitter in parasympathetic nerves. As expected, Mch (100 μM) rapidly increased [Ca²⁺]ᵢ as fluo-3 fluorescence increased by 162±26% (ΔF/F₀), and concurrently caused cell shortening by 49±8% (n=21; FIG. 6A and FIG. 6B). Strikingly, chloroquine (1 mM) almost completely reversed this [Ca²⁺]ᵢ increase (i.e., bringing [Ca²⁺]ᵢ down to a level only 15±2% higher than pre-stimulation levels, n=12, P<0.01 Mch vs Mch+ chloroquine). The reversal of the increase in [Ca²⁺]ᵢ was closely associated with relaxation in ASM cells from both mouse (back to 89±7% of the pre-stimulation length; FIG. 6B) and human (back to 94±5% of the control length, FIG. 12A). Denatonium (1 mM) generated similar effects on [Ca²⁺]ᵢ and cell shortening in response to Mch in mouse ASM cells (n=9).

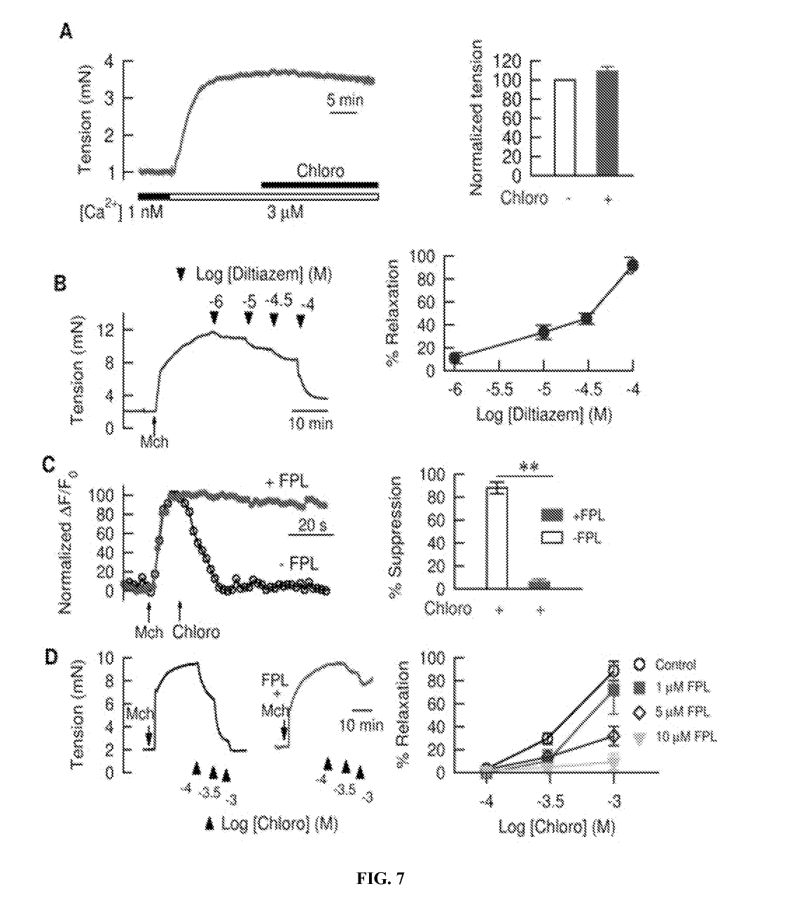

The inverse relationship between changes in [Ca²⁺]_{I} and the resulting cell length (i.e., lowering [Ca²⁺]_{I} results in cell lengthening) in response to bitter tastants indicates that bitter tastants reduce [Ca²⁺]ᵢ, leading to bronchodilation. If this is the case, one would expect that bitter tastant-induced bronchodilation could be prevented if [Ca²⁺]ᵢ was clamped to a physiologically high level. To test this possibility, we used staphylococcal α-toxin (16,000 μ/ml) to make the ASM membrane permeable to ions such that the intracellular [Ca²⁺]ᵢ could be controlled at will. A major advantage of using this toxin is that it does not damage the cells; thus signaling processes such as the G-protein-coupled receptor mediated signaling remain intact. (Kitazawa, et al. 1989 J Biol Chem 264:5339-5342.) As shown in FIG. 7A, raising [Ca²⁺]ᵢ to 3 caused a robust increase in tension in mouse airway. More importantly, at this fixed [Ca²⁺]ᵢ level, denatonium, chloroquine and quinine (all at 1 mM) failed to relax ASM in the time frame they would have in Mch contracted airways without α-toxin treatment. Therefore, clamping [Ca²⁺]ᵢ at μM levels can prevent bitter tastant-induced bronchodilation, strongly arguing that reduction of [Ca²⁺]ᵢ by bitter tastants is necessary for their relaxation action. These results further imply that a decrease in Ca²⁺ sensitivity is probably not a major mechanism underlying bitter tastant-induced bronchodilation.

### Bitter Tastants Inhibit L-Type VDCCs to Decrease [Ca²⁺]ᵢ Evoked by Bronchonconstrictors

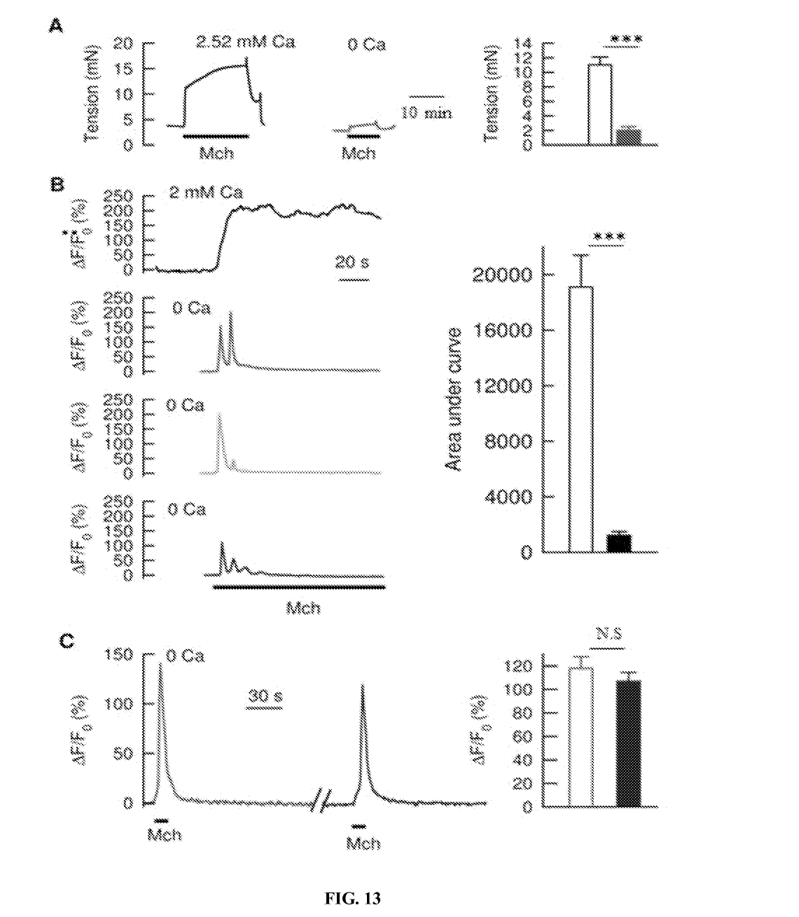

Mch activates both the M3 muscarinic acetylcholine receptor (M3R)-Gq-PLCβ-IP3 pathway and the M2 muscarinic acetylcholine receptor (M2R)-Gi/o pathway to raise [Ca²⁺]ᵢ by releasing Ca²⁺ from internal stores and inducing Ca²⁺ influx from the extracellular space (Gosens, et al. 2006 Respiratory Research 7:73; Hirota, et al. 2003 British J Anaesthesia 90:671-675; Kajita, et al. 1993 Am J of Physiology 264:L496-503; Liu, et al. 2006 Am J Physiol Lung Cell Mol Physiol 291:L281-288). It has been suggested that Ca²⁺ release from the internal stores contributes to the early phase of Mch-induced contraction, and Ca²⁺ influx via L-type voltage-dependent Ca²⁺ channels (VDCCs) is largely required to sustain elevated [Ca²⁺]ᵢ and for contraction. Indeed, the sustained contraction by Mch in mouse ASM is largely dependent on Ca²⁺ influx (FIG. 13). We established that L-type VDCCs are the major contributor to Ca²⁺ influx and sustained contraction since diltiazem, an L-type VDCC blocker, reversed Mch-induced airway force generation dose-dependently in mouse and human airways (FIG. 7B and FIG. 12B), and reversed the Mch-induced increase in [Ca²⁺] by 90.2±2.9% in single isolated mouse ASM cells (n=12 cells). Given the prominent role of L-type VDCCs in Mch-induced sustained contraction, and the fact that bronchodilation by bitter tastants acting during the sustained contractile phase (Deshpande, et al. 2010 Nat Med 16:1299-1304; Zhang, et al. 2012 Nat Med 18:648-650) has been demonstrated by us and others, we hypothesized that bitter tastants inhibit L-type VDCCs, leading to relaxation of airways precontracted by Mch. To test this possibility, it was investigated whether the L-type VDCC agonist FPL can prevent the inhibitory effect of bitter tastants on the Mch-induced [Ca²⁺]ᵢ rise and contraction. At the single cell level, 10 μM FPL can prevent chloroquine from reducing the [Ca²⁺]ᵢ increase caused by Mch (FIG. 7C). At the tissue level, FPL can prevent chloroquine from relaxing, in a dose-dependent manner, Mch precontracted mouse ASM (FIG. 7D). These results suggest that bitter tastants inhibit L-type VDCCs, which in turn leads to a decrease in [Ca²⁺]ᵢ and resulting bronchodilation.

### Bitter Tastants Reverse [Ca²⁺]ᵢ Rise and Contraction Evoked by Depolarization Activation of L-Type VDCCs

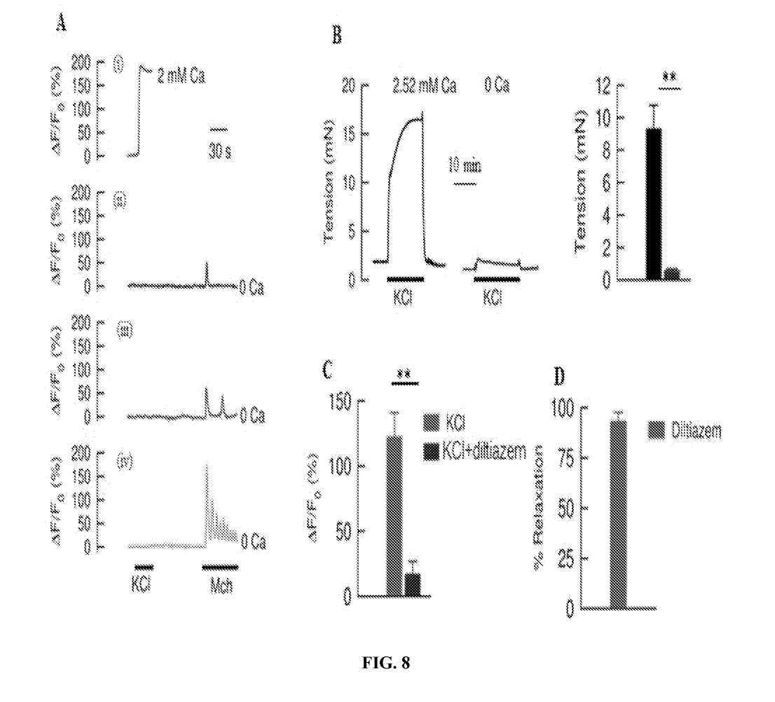

To directly examine the inhibitory role of bitter tastants on L-type VDCCs, we studied the effect of bitter tastants on KCl-induced increases in [Ca²⁺]ᵢ and contraction, and on L-type VDCC currents using patch clamp recording. KCl is a desirable bronchoconstrictor for this line of experiments because most likely it does not involve complex signaling processes (as does Mch). To test this, we compared the contraction and [Ca²⁺]ᵢ response to KCl in the presence of extracellular Ca²⁺ and in its absence. In Ca²⁺ containing medium, KCl (60 mM) induced a prominent increase in [Ca²⁺]ᵢ (FIG. 8A) and contraction (FIG. 8B). Yet in Ca²⁺ free medium, the same KCl failed to cause any increase in [Ca²⁺]ᵢ or a significant contraction (FIG. 8A and FIG. 8B). Since L-type VDCCs are the major Ca²⁺ channel for Ca²⁺ influx upon depolarization in ASM (Kotlikoff 1988 Am J Physiol 254:C793-801), the effect of diltiazem, a L-type channel blocker, on KCl-induced increase in [Ca²⁺]ᵢ and contraction was examined. It was observed that 10 μM diltiazem pretreatment reduced the KCl-induced increase in ΔF/F₀ from 122±19% to 16.8±10% (n=9, FIG. 8C); it also reversed the KCl-induced contraction by 93.1±4.8% (n=6, FIG. 8D). Therefore in mouse ASM high KCl increases [Ca²⁺]ᵢ and causes contraction by depolarizing the membrane and activating L-type VDCCs.

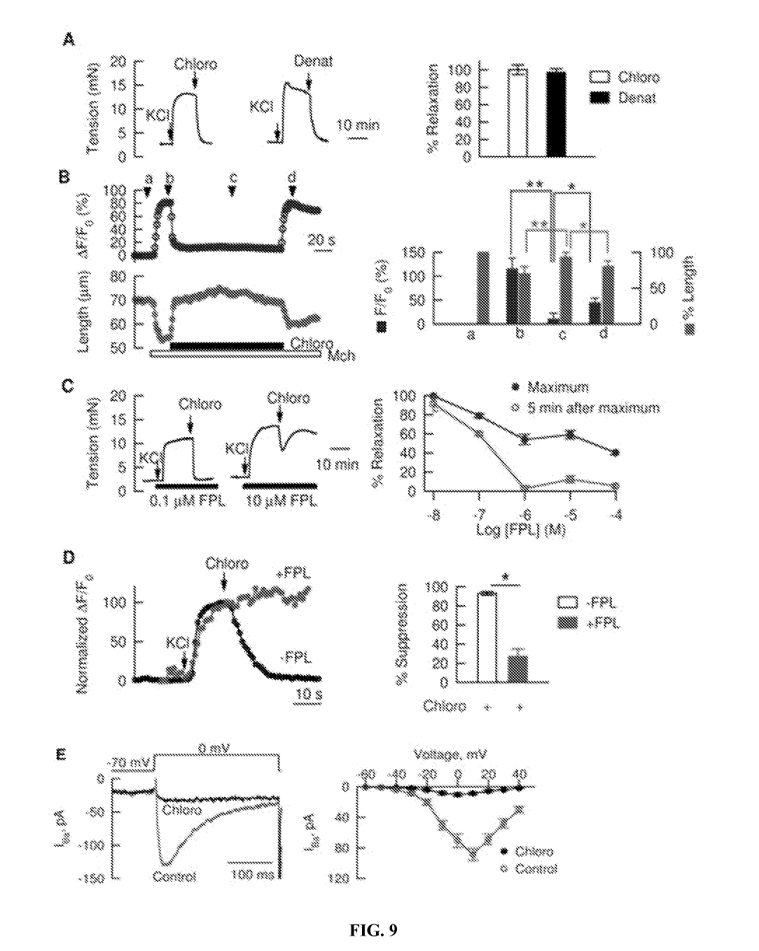

Give the action of KCl as revealed in FIG. 8, it was expected that bitter tastants would also relax ASM precontracted by KCl if bitter tastant's inhibition of L-type Ca²⁺ channels underlies its relaxation of ASM pre-contracted by Mch (FIG. 7). Indeed, it was found that 60 mM KCl caused a robust increase in tension in mouse and human airways, and this increase could be fully reversed by either chloroquine (1 mM) or denatonium (1 mM) (FIG. 9A and FIG. 12C). Similar to their effects on Mch-induced responses (FIG. 6B), chloroquine reversed the KCl-induced increase in [Ca²⁺]ᵢ and cell shortening (FIG. 9B, n=7). Moreover, FPL dose-dependently reversed chloroquine-induced relaxation in ASM pre-contracted by KCl (60 mM, FIG. 9C), and prevented the reduction of [Ca²⁺]ᵢ by chloroquine in cells stimulated by KCl (FIG. 9D). Finally, patch clamping recordings directly showed that chloroquine (1 mM) fully inhibited L-type Ca²⁺ channel currents within 2 minutes of application (FIG. 9E).

### Gβγ Activation Mediates Bitter Tastant Suppression of the Rise in [Ca²⁺]ᵢ Evoked by Activation of L-Type VDCCs

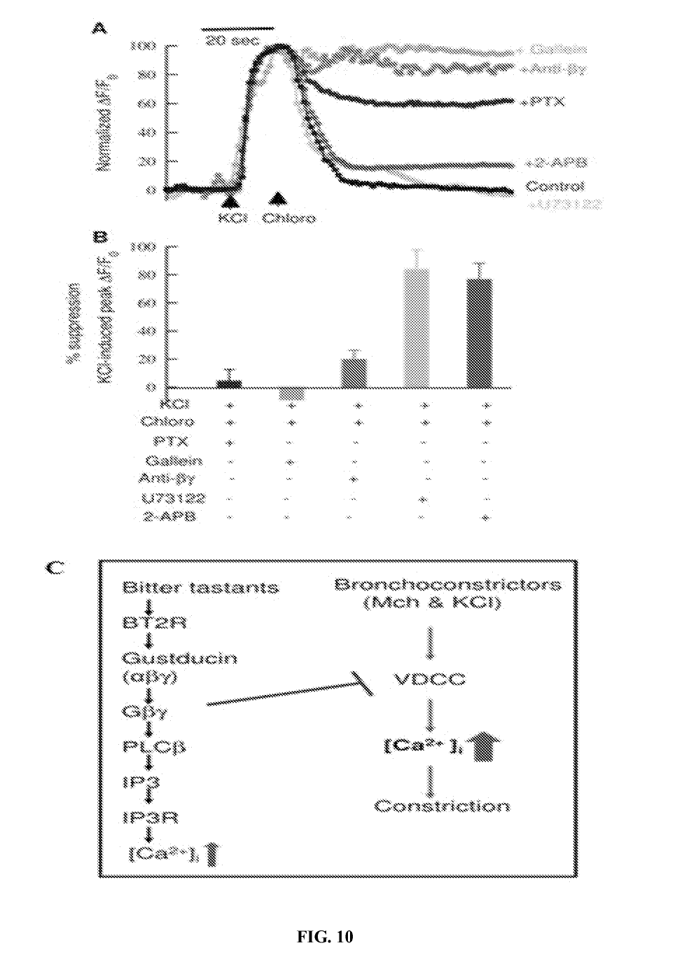

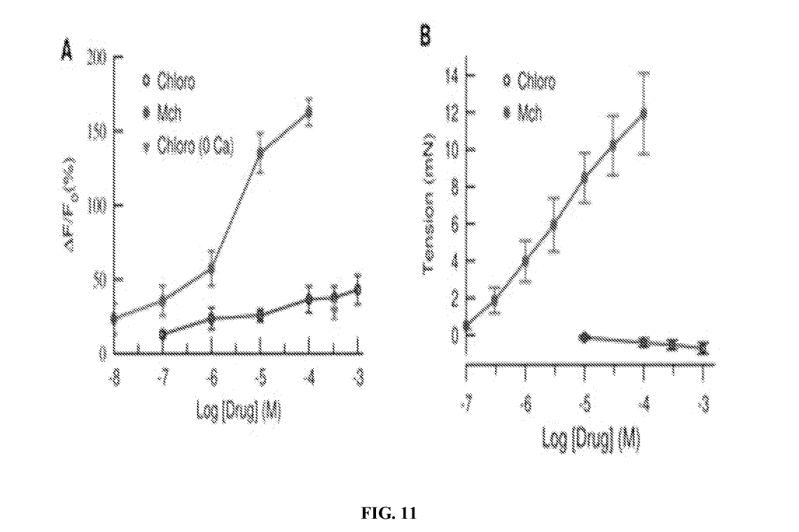

To address the signaling basis underlying bitter tastant inhibition of L-type VDCCs, we studied the impact of perturbing TAS2R signaling on bitter tastant-induced reversal of the [Ca²⁺]ᵢ increase in response to KCl. Pretreatment with PTX at 1 μg/ml for 6-8 hours prevented chloroquine-induced reversal of the KCl-induced increase in [Ca²⁺]ᵢ as did gallein (20 μM) and Anti-βγ, a Gβγ blocking peptide (1 μM) (FIG. 10). However, U73122 and 2-ABP, at the concentrations that block the bitter tastant-induced increase in [Ca²⁺]ᵢ in resting cells (FIG. 5), failed to alter chloroquine's ability to reverse a KCl-induced increase in [Ca²⁺]ᵢ (FIG. 10). These results indicate that activation of Gβγ but not PLCβ and IP3R is required for bitter tastant-induced inhibition L-type VDCCs.

When administered alone to ASM cells at rest, bitter tastants activate the canonical TAS2R signaling pathway to modestly raise [Ca²⁺]ᵢ (FIG. 10C) without affecting the contraction. Yet when applied in the presence of the bronchoconstrictor Mch, they inhibit L-type VDCCs, leading to a reversal of both the evoked [Ca²⁺]ᵢ rise and the contraction (FIG. 10C). Remarkably, both types of Ca²⁺ signals require Gβγ, while only the increase in resting [Ca²⁺]ᵢ depends on PLCβ2 activation and IP3 generation.

Bitter Tastants Reverse Contractile Agonist-Induced Increase in [Ca²⁺]ᵢ in Isolated Smooth Muscle Cells from Mouse Internal Anal Sphincter

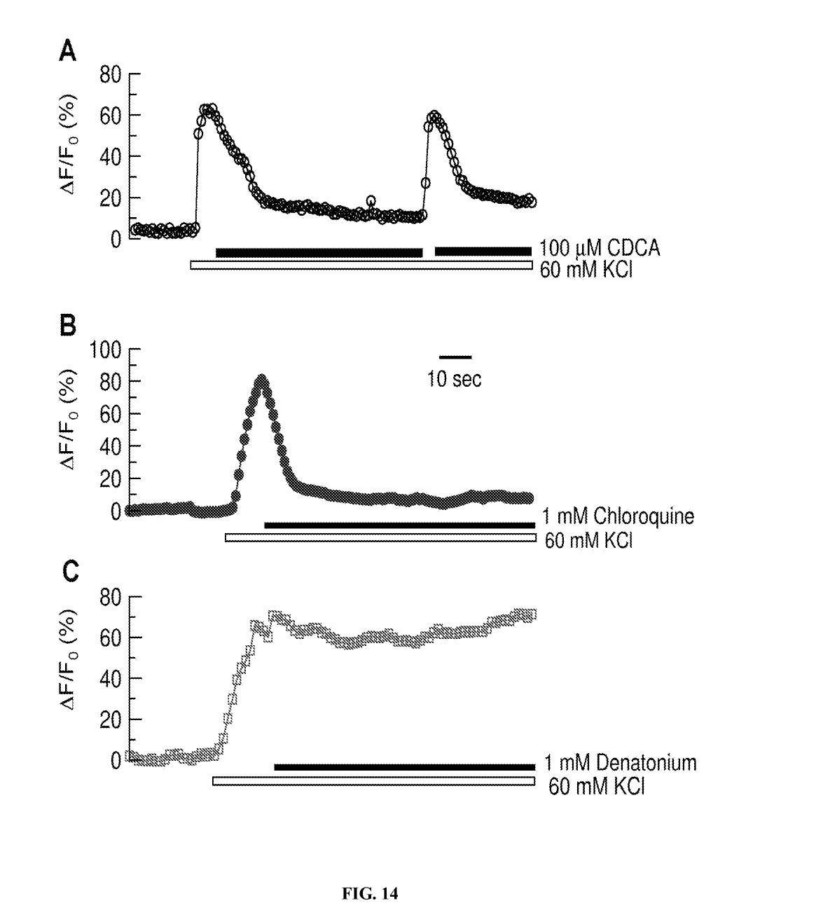

As shown in FIG. 14, bitter tastants reverse contractile agonist-induced increase in [Ca²⁺]ᵢ in isolated smooth muscle cells from mouse internal anal sphincter. FIG. 14A shows that CDCA (100 μM) reversibly inhibited the increase in [Ca²⁺]ᵢ induced by 60 mM KCl. FIG. 14B shows that chloroquine (1 mM) fully reversed the increase in [Ca²⁺]ᵢ induced by 60 mM KCl. Denatonium (1 mM) did not affect the [Ca²⁺]ᵢ response to 60 mM KCl (FIG. 14C).

### Effects of Bitter Tastants on Smooth Muscle

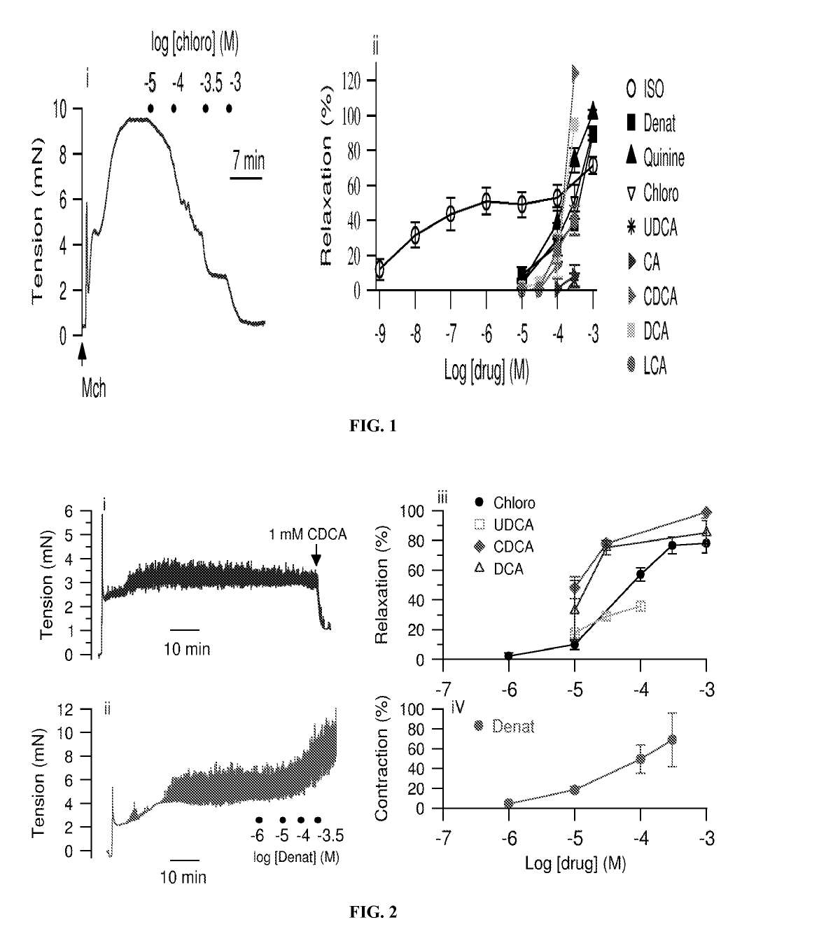

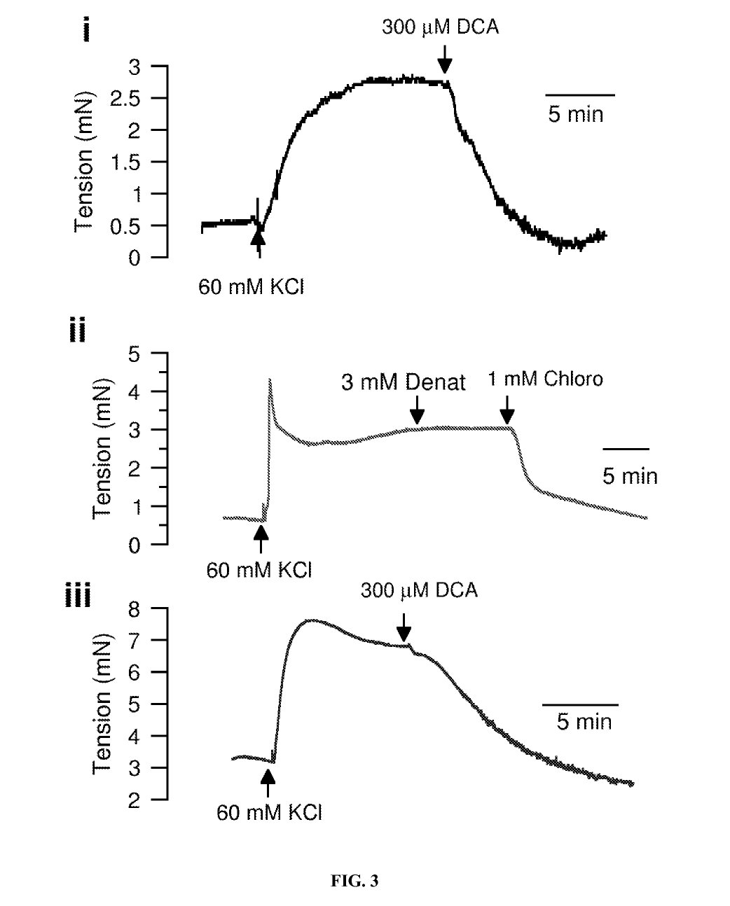

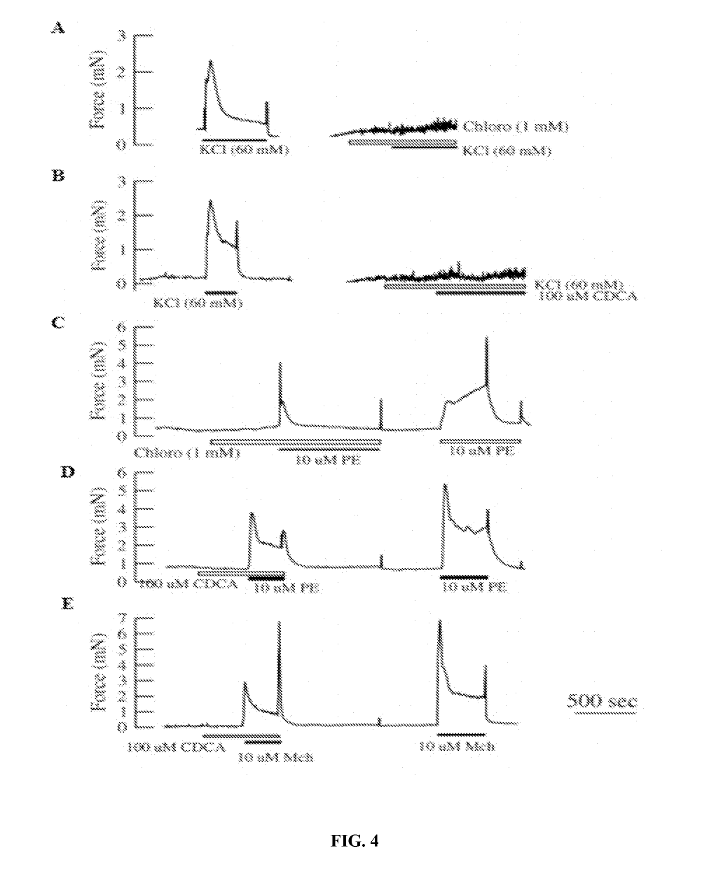

Tests showed that bitter compounds including two bile acids (e.g., DCA and CDCA shown below) can fully relax smooth muscle from airways, internal anal sphincter, pulmonary artery, and urethra from mouse, and smooth muscle from airways and pulmonary artery from human (FIGS. 1-4).

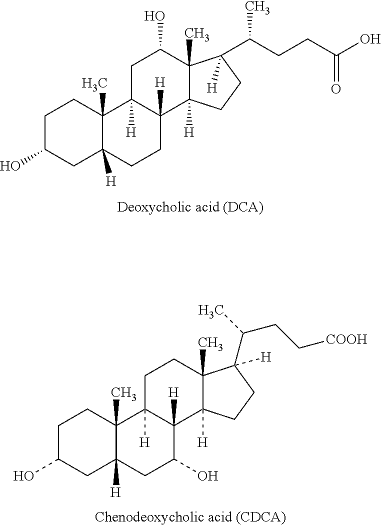

Results also showed that some bitter compounds relax airway smooth muscle, yet contract internal anal sphincter smooth muscle (FIG. 2). These compounds may be used to treat smooth muscle disorders due to weak contraction (e.g., hypotension, urinary incontinence, fecal incontinence and constipation).

### Method for Screening Compounds and Identifying Smooth Muscle Relaxants

Based on the molecular mechanisms proposed herein by which bitter tastants relax smooth muscle, a method was developed for screening agents for smooth muscle relaxants. Results showed that bitter tastants reverse bronchoconstrictor-induced increase in intracellular Ca concentration ([Ca²⁺]ᵢ) is the underlying signal for their relaxation. This phenomon can be demonstrated robustly and quantatively by simulateous measurement of [Ca²⁺]ᵢ and cell length change at single cell level. The screening method disclosed herein is well suited for identifying relaxants from among the many thousands of available bitter tastants.

### Materials and Methods

### Animal Tissue Handling

Experimental protocols for animal research were approved by the Institutional Animal Care and Use Committees at the University of Massachusetts Medical School (protocol A-1473 to R.Z).

### Isolation of Mouse Airway Smooth Muscle Cells

C57/BL6 mice from 7 to 12 weeks of age were anesthetized with intraperitoneally injected pentobarbitone (50 mg kg⁻¹), and the trachea and mainstem bronchi were quickly removed and placed in a pre-chilled dissociation solution consisting of (in mM): 135 NaCl, 6 KCl, 5 MgCl₂, 0.1 CaCl₂, 0.2 EDTA, 10 HEPES, and 10 Glucose (pH 7.3). The tracheas and bronchi were dissected free from the surface of the connective tissue. The tissue was incubated in the dissociation medium containing papain 30 unit/ml, 1 mM DTT, and 0.5 mg/ml BSA, at 35° C. for 30 min, and then transferred to a dissociation medium containing 3 unit/ml collagenase F and 0.5 mg/mL BSA, and incubated at 35° C. for another 15 min to produce isolated ASM cells. Finally, the tissue was agitated with a fire polished wide-bore glass pipette to release the cells.

### Mouse Airway Smooth Muscle Contraction Bioassay

C57/BL6 mice at 7-12 weeks of age were sacrificed and the entire respiratory trees were rapidly removed and immersed in Krebs physiologic solution containing (in mM) 118.07 NaCl, 4.69 KCl, 2.52 CaCl₂, 1.16 MgSO₄, 1.01 NaH₂PO₄, 25 NaHCO₃, and 11.10 glucose. Trachea and bronchi were isolated and cut into rings (4 mm in length). The rings were mounted on a wire myograph chamber (Danish Myo Technology, Aarhus, Denmark), and a PowerLab recording device (AD Instruments) was used to record isometric tension. The ring preparations with zero tension were immersed in 5 ml of Krebs physiologic solution, bubbled with 95% 02 and 5% CO2 at 37° C. The basal tones were set at the level of approximately 2 mN. The order and treatment time of agonists and antagonists are indicated in the figure captions.

### Lung Tissue

Human lung tissue was obtained (with informed consent) from patients undergoing surgery (lobectomy) for lung cancer at the Department of Surgery and the Department of Pathology at the Univ. of Massachusetts Memorial Med Ctr (Worcester, Mass.). The tumors were identified as nonsmall cell carcinoma (adenocarcinoma, squamous cell carcinoma). Intrapulmonary airways were dissected out and cleaned free of the connective tissues. These airways were either cut into the rings (4 mM in length) for force measurements the same as for mouse airway tissues, or digested with the same enzymes, dissociation medium and isolation procedure as for single mouse ASM cells. The experimental protocols on human tissues were approved by the Committee for Protection of Human Subjects in Research at the University of Massachusetts Medical School (Protocol 13590 to R.Z).

### Measurement of Global [Ca²⁺]ᵢ and Ca²⁺ Sparks

Fluorescence images using fluo-3 as a calcium indicator were obtained using a custom-built wide-field digital imaging system. The camera was interfaced to a custom made inverted microscope, and the cells were imaged using either a 20× Nikon 1.3 NA for global [Ca²⁺] measurement or a 60× Nikon 1.4 NA oil for Ca²⁺ spark measurement. The 488 nm line of an Argon Ion laser provided fluorescence excitation, with a shutter to control exposure duration, and emission of the Ca²⁺ indicator was monitored at wavelengths >500 nm. The images were acquired at the speed of either 1 Hz for global [Ca²⁺] measurement or 50 Hz for Ca²⁺ spark measurement. Subsequent image processing and analysis was performed off line using a custom-designed software package, running on a Linux/PC workstation. [Ca²⁺]ᵢ was represented as ΔF/F₀*100 with F calculated by integrating fluo-3 over entire cells for global [Ca²⁺], or just the value at the brightest pixel (i.e., epicenter pixel) for Ca²⁺ sparks.

### Patch-Clamp Recording

Membrane currents were recorded with an EPC10 HEKA amplifier under perforated whole-cell patch recording configuration. The extracellular solution contained (in mM): NaCl 126, Tetraethylammonium Cl 10, BaCl₂ 2.2, MgCl₂ 1, Hepes 10, and glucose 5.6; pH adjusted to 7.4 with NaOH. The pipette solution contained (in mM): CsCl 139, MgCl₂ 1, Hepes 10, MgATP 3, Na₂ATP 0.5; pH adjusted to 7.3 with KOH; amphotericin B was freshly made and added to the pipette solution at a final concentration of 200 μg/ml. Whole-cell Ba²⁺ currents were evoked by step depolarization with 300 ms duration every 10 s from a holding potential of −70 mV at a 10 mV increment. Currents were leak corrected using a P/4 protocol.

### Measurement of Cell Shortening

Myocytes were placed into a recording chamber superfused with the bath solution for patch clamp experiments at room temperature. Cells loaded with Fluo-3 were imaged using a custom-built wide-field digital imaging system and their lengths were determined using custom software to manually trace down the center of the cell.

RT-PCR to Detect mRNA

The connective tissues in trachea and mainstem bronchi were carefully removed and the ASM were then quickly frozen in dry ice. The total RNA of the ASM was isolated with the TRIzol™ (Invitrogen) method following the manufacturer's guidelines; and cDNA was synthesized using extracted RNA with an Omniscript Reverse Transcription Kit (Qiagen). The specific primers, synthesized by Invitrogen, are listed in Table 1. β-actin was used as a positive control and the absence of DNA as a negative control, and the PCR reaction was carried out in a PCR mastercycler.

Reagents and their Application

All chemicals, except fluo-3 (Invitrogen Co, San Diego, Calif., USA), gallein (Tocris Bioscience, Bristol, United Kingdom), and Anti-βγ blocking peptide (AnaSpec, Fremont, Calif., USA), were purchased from Sigma-Aldrich Co. (St. Louis, Mo., USA). For single cell studies, agonists and antagonists were applied locally to cells via a picospritzer at a constant pressure, so that the duration of its action and concentration could be controlled easily.

### Statistics

Unless stated otherwise, data are reported as mean±s.e.m and n means numbers of cells or trachea and mainstem bronchi. Statistical analysis of differences was made with Student's paired or unpaired t-test and the significance level was set at p<0.05.

TABLE 1
Primers for RT-PCR

| Gene | Forward | Reverse |
|---|---|---|
| TAS2R107 | TTCCAACTCTGTATTTCTCT | TAATTTTTCCGCTGGTGGA |
| | GGC | |
| TAS2R108 | CTAATTTTCAACACCCAGTG | CCCAATTATGTGTTCAGGA |
| α- | TGGTTACAGCAAACAAGAAT | TTCAAAGCAGGCTTGGATT |
| gustducin | GC | |
| Gβ3 | CAGGACAGCAGAAGACAGTG | GTCATCTGAGCCAGTGCAG |
| Gγ13 | CCCAGCCTCACTCCACAGAT | CCTCTTGAAGGCCAGTTGG |
| PLCβ2 | ATGCAGCAGAACATGGCACT | CCAGCTCAGGCATCAAGAT |
| β-actin | AGGCCAACCGTGAAAAGAT | AGAGCATAGCCCTCGTAGA |

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A method for identifying a candidate compound for treating or preventing a smooth muscle disorder, comprising:
contacting a test compound with a cell of a smooth muscle tissue or organ; and
measuring the intracellular Ca²⁺ concentration before and after contacting the test compound, whereby a decrease of 30% or greater after contacting the test compound is indicative of the activity of the test compound.

2. The method of claim 1, wherein the test compound is a bitter tastant.

3. The method of claim 1 further comprising after contacting a test compound,
measuring the cell length before and after contacting with the test compound, wherein an increase of 20% or greater is indicative of the activity of the test compound.

4. The method of claim 1, wherein the smooth muscle tissue or organ is part of the respiratory tract, part of a blood vessel, part of the gastrointestinal tract, part of the urinary tract, part of internal anal sphincter, or part of pulmonary artery.

5-10. (canceled)

11. A method for treating or preventing a smooth muscle disorder in a mammal, including human, comprising administering to a subject in need thereof a therapeutically effective amount of a pharmaceutical composition comprising a compound having the structural formula of (I): 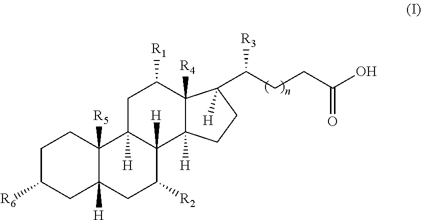
or a pharmaceutically acceptable salt, ester or prodrug thereof,
wherein each of R₁, R₂, R₃, R₄, R₅ and R₆ is independently selected from hydrogen, OH, alkyl, alkoxy, and halogen; n is an integer from 0 to about 4.

12. The method of claim 11, wherein the compound has the structural formula:

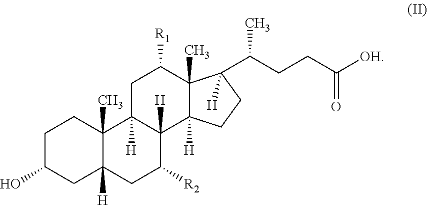

13. The method of claim 11, wherein the smooth muscle disorder is an airway obstructive disease selected from asthma and chronic obstructive pulmonary disease (COPD).

14. (canceled)

15. (canceled)

16. The method of claim 11, wherein the smooth muscle disorder is anal sphincter disorder, urethral obstruction, a disorder associated with cystic fibrosis, a disorder associated with fecal incontinence and constipation, or a disorder is associated with pulmonary hypertension.

17-20. (canceled)

21. The method of claim 12, wherein R₁ is hydrogen or OH.

22. (canceled)

23. The method of claim 12, wherein R₂ is hydrogen or OH.

24. (canceled)

25. The method of claim 12, wherein the compound is:

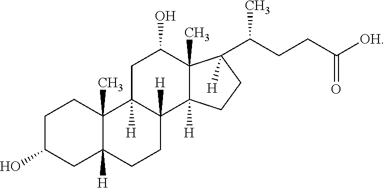

26. The method of claim 12, wherein the compound is:

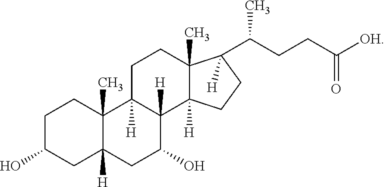

27. A pharmaceutical composition for treating or preventing a smooth muscle disorder in a mammal, including human, comprising a therapeutically effective amount of a pharmaceutical composition comprising a compound having the structural formula of (I): 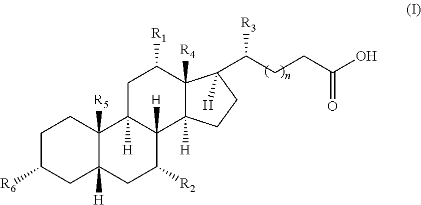
or a pharmaceutically acceptable salt, ester or prodrug thereof,
wherein each of R₁, R₂, R₃, R₄, R₅ and R₆ is independently selected from hydrogen, OH, alkyl, alkoxy, and halogen; n is an integer from 0 to about 4.

28. The pharmaceutical composition of claim 27, wherein the compound has the structural formula:

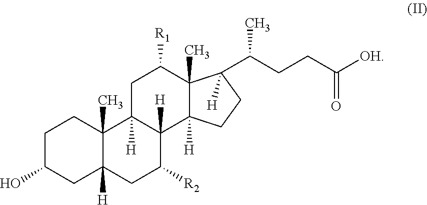

29. The pharmaceutical composition of claim 27, wherein the smooth muscle disorder is an airway obstructive disease selected from asthma and chronic obstructive pulmonary disease (COPD).

30. (canceled)

31. (canceled)

32. The pharmaceutical composition of claim 27, wherein the smooth muscle disorder is anal sphincter disorder, urethral obstruction, a disorder associated with cystic fibrosis, a disorder associated with fecal incontinence and constipation, or a disorder is associated with pulmonary hypertension.

33-36. (canceled)

37. The pharmaceutical composition of claim 28, wherein R₁ is hydrogen or OH.

38. (canceled)

39. The pharmaceutical composition of claim 28, wherein R₂ is hydrogen or OH.

40. (canceled)

41. The pharmaceutical composition of claim 28, wherein the compound is:

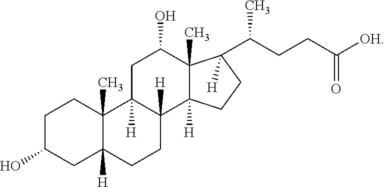

42. The pharmaceutical composition of claim 28, wherein the compound is: [chemical structure]
